(19) United States
(12) Patent Application Publication
(10) Pub. No.: US 2025/0066284 A1
(43) Pub. Date: Feb. 27, 2025

(54) PHOTOPOLYMERIZATION INITIATORS AND THEIR USE

(71) Applicant: UNIVERSITY OF IOWA RESEARCH FOUNDATION, Iowa City, IA (US)

(72) Inventors: C. Allan Guymon, Iowa City, IA (US); Huayang Fang, Iowa City, IA (US)

(73) Assignee: UNIVERSITY OF IOWA RESEARCH FOUNDATION, Iowa City, IA (US)

(21) Appl. No.: 18/813,869

(22) Filed: Aug. 23, 2024

Related U.S. Application Data
(60) Provisional application No. 63/534,535, filed on Aug. 24, 2023.

(51) Int. Cl. C07C 205/56; C08F 2/50

(52) U.S. Cl. CPC C07C 205/56; C08F 2/50; C07C 2602/10; C07C 2603/24

(57) ABSTRACT

Disclosed herein are photopolymerization initiators of formula I.

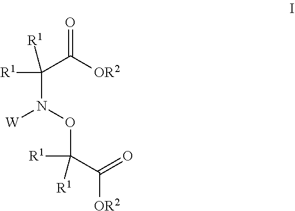

or a salt thereof. Also disclosed are compositions comprising the photopolymerization initiators of formula I and methods of using the photopolymerization initiators of formula I.

### CROSS-REFERENCE TO RELATED APPLICATIONS

This application claims priority to U.S. Provisional Application No. 63/534,535 that was filed on Aug. 24, 2023. The entire content of the application referenced above is hereby incorporated by reference herein.

### BACKGROUND OF THE INVENTION

Controlled radical polymerization (CRP) has grown considerably over the past three decades due to the demand to develop well-defined (pre)polymers such as block copolymers (1,2). Typical CRP process relies on the formation of persistent radicals capable of undergoing a series of reversible degenerative or reversible deactivation reactions with propagating radicals, leading to controlled chain propagation (3,4). Atom transfer radical polymerization (ATRP), reversible addition-fragmentation chain transfer (RAFT), and nitroxide mediated polymerization (NMP) are some of the more popular CRP techniques that have been employed to prepare polymers with defined molecular weight and low polydispersity (5-9). NMP technology, in particular, is a straightforward and simple means to develop polymers with specific architectures without additional metal catalysts or post-purification treatment (10,11) Heat and light are often used as stimuli to activate the NMP mechanism (12-14). Compared to the thermal NMP process, the photo-induced NMP has been recognized as a green method to form polymer materials due to low energy consumption and no need for solvent (15). Most photo-induced NMP reactions are conducted at room temperature which prevents undesired side reactions during polymerization. In addition, using light to activate NMP also provides good spatial and temporal control (16).

In the photo-induced NMP process, the alkoxyamines and/or alkoxyamine-derivatives are often utilized to generate persistent radials that facilitate control during polymerization by trapping and releasing propagating radicals to undergo the reversible activation and deactivation processes (15,17,18). A typical NMP mechanism is shown in FIG. 1. After initiation, a carbon-centered propagating radical and nitroxide radical are dissociated through the C—ON bond cleavage of alkoxyamine compounds. The nitroxide radicals, as the persistent radicals, are very stable and do not react with monomers/oligomers but favor the recombination reaction with propagating radicals. On the other hand, the carbon-centered radicals act like regular free radicals and propagate with unreacted monomers/oligomers. Due to the presence of nitroxide radicals, the propagating radicals and active polymer chains are temporarily terminated and then re-activated during chain propagation, allowing each polymer chain an equal opportunity to grow. As a result, the controlled polymerization reduces the irreversible termination events which are typically seen from free radical polymerization (4). Meanwhile, compared to the RAFT-based CRP process, the NMP process also prevents unfavored chain rearrangement (19, 20).

Taking advantage of NMP-controlled polymerization, significant research has focused on developing photo-inducible NMP-based initiators. The 2,2,6,6-tetramethyl-1-piperidinyloxy (TEMPO)- and TEMPO derivative-based photosensitive alkoxyamines were first developed and tested by Scaiano and coworkers in 1997. When photosensitized by xanthone, TEMPO-based alkoxyamine homolyze to TEMPO and carbon-centered radicals (21). Based on the potential usage of TEMPO compounds for photo-induced NMP, several TEMPO-containing chromophores were synthesized and utilized as photoinitiators for methyl methacrylate (MMA) polymerization. Controlled polymerization reaction with increased molecular weight was observed (22). Goto and coworkers synthesized the TEMPO-containing quinoline chromophore initiator and showed living free-radical polymerization properties when polymerizing styrene at low temperatures (23). In order to increase the controllability during polymerization, the addition of a photo-acid generator in the photo-induced TEMPO-based NMP process was investigated. By polymerizing in MMA formulations, a narrow molecular weight distribution is exhibited for the produced poly(methyl methacrylate) (24-26). Later, several different photosensitive-nitroxides were synthesized and showed controlled polymerization evidence and narrow polydispersity when polymerizing with MMA (27,28). However, this photosensitive-nitroxide mediation process needs external initiators (e.g., conventional photoinitiator) to form bi-molecular initiator systems. Thus, developing a unimolecular alkoxyamine-based NMP photoinitiator that enables controlled radical polymerization is therefore very desirable.

Recently, a new alkoxyamine-based NMP initiator which has the chromophore group directly attached to the alkoxyl functional group was reported (29). This chromophore-containing alkoxyamine showed improved energy transfer efficiency from the chromophore moiety to the alkoxyamine compound, thereby facilitating the homolysis of the alkoxyamine. When photocuring with butyl acrylate, the 1n([M]₀/[M]) value increases linearly, and a linear relationship between molecular weight and conversion was observed. The reinitiation behavior of this NMP-based initiator was also confirmed by carrying out the two-step polymerization of n-butyl acrylate. However, a broad polydispersity is still observed indicating only a partial-controlled polymerization. Accordingly, there is a need for new photopolymerization initiators such as NMPs to prepare polymers with unique and useful properties.

### SUMMARY OF THE INVENTION

Photopolymerization initiators disclosed herein are useful for polymerizing compounds (e.g., monomers such as (meth)acrylates) including mixtures of two or more different compounds (e.g., mixtures of two or more different oligomers/monomers such a mixture of two or more different (meth)acrylates) to provide polymers. The resulting polymers have unique and useful properties.

Accordingly, one embodiment provides a photopolymerization initiator of formula I:

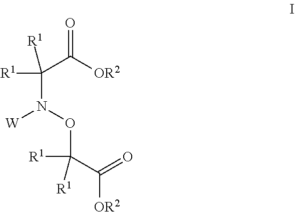

or a salt thereof, wherein:
W is an aryl, wherein the aryl is optionally substituted with one or more groups independently selected from the group consisting of halo, —OH, (C₁-C₄)alkyl, and —C(═O)Ph wherein the —C(═O)Ph is optionally substituted with halo, —OH, or (C₁-C₄)alkyl;
each R¹ is independently (C₁-C₄)alkyl;
each R² is independently (C₁-C₆)alkyl;
provided the compound is not:

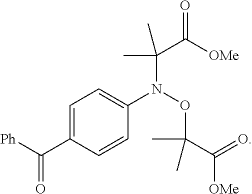 [SMILES: COC(=O)C(C)(C)ON(c1ccc(C(=O)c2ccccc2)cc1)C(C)(C)C(=O)OC]

One embodiment provides a photocurable composition comprising a photopolymerization initiator disclosed herein (e.g., a photopolymerization initiator of Formula I).

One embodiment provides a photocurable composition comprising a photopolymerization initiator disclosed herein (e.g., a photopolymerization initiator of Formula I) and a polymerizable compound.

One embodiment provides a method to polymerize a polymerizable compound or mixture of two or more different polymerizable compounds, comprising contacting the polymerizable compound or mixture of two or more different polymerizable compounds with a photopolymerization initiator disclosed herein (e.g., a photopolymerization initiator of Formula I).

One embodiment provides a polymer prepared by any of the methods disclosed herein.

### BRIEF DESCRIPTION OF THE FIGURES

FIG. 1 shows the mechanism of nitroxide mediated polymerization process.

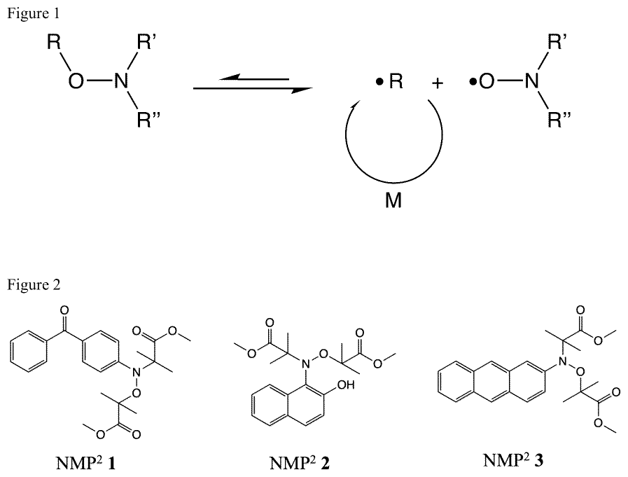

FIG. 2 shows nitroxide mediated photopolymerization (NMP²) initiators: 1 (benzophenone-derived), 2 (naphthol-derived), and 3 (anthracene-derived).

FIG. 3 shows UV-vis spectra of benzophenone derived NMP² 1, naphthol derived 2, and anthracene derived 3 initiators. Two conventional photoinitiators, including DMPA (—) and BAPO (—) were also measured in acetonitrile at room temperature. Samples are degassed with argon for 40 minutes.

FIGS. 4A-4D show spectral changes of NMP² initiator 1, 2, and 3 in acetonitrile (degassed with argon for 60 mins) under irradiation with UV light with 320-500 nm band filter at 20 mW/cm². FIG. 4A shows DMPA, FIG. 4B shows NMP² 1, FIG. 4C shows NMP² 2, and FIG. 4D shows NMP² 3.

FIGS. 5A-5C show absorbance changes of NMP² initiators. FIG. 5A shows NMP² 1 at λ=320 nm, FIG. 5B shows NMP² 2 at λ=335 nm, and FIG. 5C shows NMP² 3 at λ=260 nm. All NMP² initiators were dissolved in acetonitrile (degassed with argon) under irradiation with UV light equipped with 320-500 nm band filter at 20 mW/cm².

FIG. 6 shows double bond conversion as function of curing time for the polymerization of monofunctional hexyl acrylate containing 0.5 mol % DMPA or 1.0 mol % NMP² initiators. Both formulations were polymerized at 20 mW/cm² using UV light equipped with a broadband filter (320-500 nm).

FIGS. 7A-7C show the absorbance changes of NMP² initiators. FIG. 7A shows NMP² 1 at λ=320 nm, FIG. 7B shows NMP² 2 at λ=335 nm, and FIG. 7C shows NMP² 3 at λ=260 nm with inert atmosphere (solid line) and in-air (dashed line). All NMP² initiators were dissolved in acetonitrile (degassed with argon) under irradiation with UV light equipped with the 320-500 nm band filter at 20 mW/cm² FIGS. 8A-8C show pseudo-first-order plots for the poly(hexyl acrylate) systems initiated with NMP² initiators. FIG. 8A shows NMP² 1; FIG. 8B shows NMP² 2; and FIG. 8C shows NMP² 3. The concentration of NMP² initiator is fixed at 1.0 mol %.

FIGS. 9A-9D show the molecular weight as a function of conversion for homo-photopolymerization of hexyl acrylate formulation initiated with FIG. 9A DMPA; FIG. 9B NMP² 1; FIG. 9C NMP² 2; and FIG. 9D NMP² 3. The concentration of NMP² initiator is 1.0 mol % while the DMPA concentration is 0.5 mol %. Both formulations were polymerized at 20 mW/cm² using UV light equipped with a broadband filter (320-500 nm) under N₂ atmosphere.

FIG. 10 shows double bond conversion as a function of curing time for DMPA and NMP²-based alkoxyamines polymerization: DMPA (⋅⋅), 1, 2, and 3. All NMP²-initiated formulations were polymerized with 1.0 mol % NMP² initiators while the control sample contains 0.5 mol % of DMPA. Samples were photocured with 20 mW/cm² light using a 320-500 nm bandpass filter.

FIGS. 11A-11D shows real-time rheology measurement of storage moduli (G′) and loss moduli (G″) for the DMPA- and NMP² initiator acrylate formulations. The dashed line indicates gelation time (t_{ge1}). Samples were photocured under UV light with 500 mW/cm² using a 320-500 nm bandpass filter. FIG. 11A shows DMPA, FIG. 11B shows NMP² 1, FIG. 11C shows NMP² 2, and FIG. 11D shows NMP² 3.

FIG. 12 shows The normal force as a function of curing time is monitored by the photorheolometry for formulations initiated by DMPA, and NMP² initiators 1, 2, and 3. Samples were photocured under UV light with 500 mW/cm² using a 320-500 nm bandpass filter.

FIG. 13 shows the storage modulus as a function of temperature for NMP² initiator 1, 2, and 3. A formulation containing DMPA (—) was also tested as the control. Samples were photocured under UV light with 20 mW/cm² using a 320-500 nm bandpass filter.

FIG. 14 shows tan delta as a function of temperature using DMPA, and NMP² 1, 2, and 3 as photoinitiators for Ebecryl 605/40. Samples were photocured under UV light with 20 mW/cm² using a 320-500 nm bandpass filter.

FIG. 15 shows bending stress as a function of deflection for NMP² initiators 1, 2, and 3. A formulation containing DMPA (—) was also tested as the control. Samples were photocured under UV light with 20 mW/cm² using a 320-500 nm bandpass filter.

FIGS. 16A-16C show stress-strain properties. FIG. 16A shows film stress as a function of strain for acrylate oligomer mixture (Ebecryl 605/40) initiated with DMPA (—), and NMP² 1, 2, and 3. FIG. 16B shows strain to break value. FIG. 16C shows tensile modulus for DMPA and NMP² initiator modified polymer films. Samples were photocured under UV light with 20 mW/cm² using a 320-500 nm bandpass filter.

FIG. 17 shows tensile toughness of epoxy acrylate formulations photoinitiated with DMPA (grey), and alkoxyamine initiator 1 (red), 2 (green), and 3 (blue). All samples were photocured under UV light with 20 mW/cm² using a 320-500 nm bandpass filter. Each experiment was conducted 4 times with the error bars representing standard deviation.

### DETAILED DESCRIPTION

Disclosed here are chromophore-derived alkoxyamine-based nitroxide-mediated photopolymerization (NMP²) initiators, including naphthol-derived alkoxyamine 2, and anthracene-derived alkoxyamine 3. The chromophore group (e.g., aryl moiety) inparts different properties to the photoiniator. Accordinly, the UV-Vis properties, photolysis properties and the photo-decay behavior for each NMP² initiator were investigated. In addition, the controlled polymerization behavior of these NMP² initiators were also evaluated.

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

The term “(Cₐ-C_{b})alkyl” wherein a and b are integers refers to a straight or branched chain hydrocarbon alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “aryl” as used herein refers to a single aromatic ring or a multiple condensed ring system wherein the ring atoms are carbon. For example, an aryl group can have 6 to 10 carbon atoms, or 6 to 12 carbon atoms, or 6 to 14 carbon atoms. Aryl includes a phenyl radical. Aryl also includes multiple condensed ring systems (e.g., ring systems comprising 2 of 3 rings) having about 9 to 14, or 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Photopolymerization Initiators

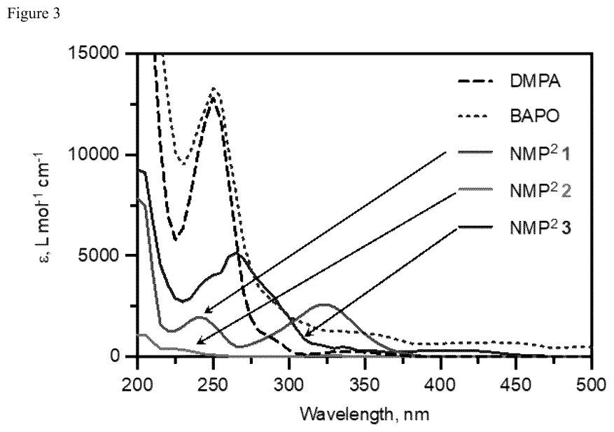

The photopolymerization initiators disclosed herein are chromophore-derived alkoxyamine-based nitroxide-mediated photopolymerization (NMP²) initiators such as the naphthalene-derived alkoxyamine 2, and the anthracene-derived alkoxyamine 3 (FIG. 2) One embodiment provides a photopolymerization initiator of formula I.

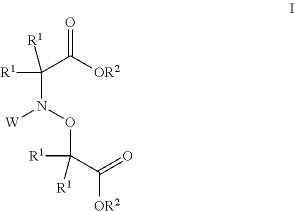

or a salt thereof, wherein:
W is an aryl, wherein the aryl is optionally substituted with one or more groups independently selected from the group consisting of halo, —OH, (C₁-C₄)alkyl, and —C(═O)Ph wherein the —C(═O)Ph is optionally substituted with halo, —OH, or (C₁-C₄)alkyl;
each R¹ is independently (C₁-C₄)alkyl;
each R² is independently (C₁-C₆)alkyl;

In one embodiment, the photopolymerization initiator of formula I is not:

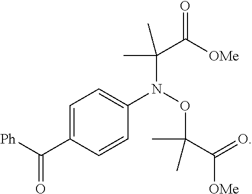 [SMILES: COC(=O)C(C)(C)ON(c1ccc(C(=O)c2ccccc2)cc1)C(C)(C)C(=O)OC]

In one embodiment, the photopolymerization initiator of claim 1, wherein each R¹ is methyl.

In one embodiment, the photopolymerization initiator of claim 1 or claim 2, wherein each R² is methyl.

In one embodiment, W is naphthalenyl or anthracenyl, wherein the naphthalenyl (naphthenyl) or anthracenyl is optionally substituted with one or more groups independently selected from the group consisting of halo, —OH, and (C₁-C₄)alkyl.

In one embodiment, W is naphthalenyl or anthracenyl, wherein the naphthalenyl or anthracenyl is optionally substituted with one or more —OH.

In one embodiment, the photopolymerization initiator is:

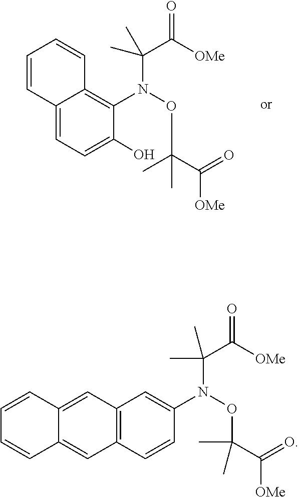 [SMILES: COC(=O)C(C)(C)ON(c1c(O)ccc2ccccc12)C(C)(C)C(=O)OC ; COC(=O)C(C)(C)ON(c1ccc2cc3ccccc3cc2c1)C(C)(C)C(=O)OC]

### Polymerizable Compounds

As used herein the term polymerizable compounds refers to any compound (e.g., monomer, oligomer) that can be polymerized with the photopolymerization initiators (e.g., NMP²) described herein. It is to be understood that the compounds being polymerized can be a plurality of the same compound or the compounds being polymerized can be a plurality of different compounds. In general, the polymerizable compounds are polymerized through a radical process that is initiated with light (e.g., UV light) in the presence of the photopolymerization initiator.

In one embodiment the polymerizable compound(s) comprises a site of unsaturation.

In one embodiment the polymerizable compound(s) comprises an unsaturated carbon-carbon double bond.

In one embodiment the polymerizable compound(s) is an acrylate or diacrylate monomer or mixture thereof.

In one embodiment the acrylate monomer is an alkyl acrylate (e.g., hexyl, pentyl, butyl, propyl ethyl, and methyl acrylate), aliphatic urethane diacrylate, bisphenol A epoxy diacrylate, alkyl diacrylate (hexanediol, decanediol), poly(ethylene glycol) acrylate, poly(ethylyene glycol) diacrylate or tri(propylene glycol) diacrylate.

In one embodiment the polymerizable compound(s) is a methacrylate or dimethacrylate monomer or mixture thereof.

In one embodiment the acrylate monomer is an alkyl methacrylate (e.g., hexyl, pentyl, butyl, propyl ethyl, and methyl methacrylate), aliphatic urethane dimethacrylate, bisphenol A epoxy dimethacrylate, alkyl dimethacrylate (hexanediol, decanediol), poly(ethylene glycol) methacrylate, poly(ethylyene glycol) dimethacrylate or tri(propylene glycol) dimethacrylate.

In one embodiment the polymerizable compound(s) is an acrylamide or bisacrylamide monomer or mixture thereof.

In one embodiment the acrylamide monomer is an alkyl acrylamide (e.g., n-isopropyl, methyl acrylamide) or methylene bisacrylamide.

### Polymers

One embodiment provides a polymer obtained by any of the methods as described herein.

Compared to polymers prepared through conventional free radical photopolymerization, the use of this NMP² as photopolymerization initiator enable the reversible termination with propagating radicals to mediate chain propagation during photopolymerization process. Observed delayed gelation ensures enhanced chain relaxation, which in turn reduces both shrinkage and shrinkage stress in acrylate polymer networks. As a result, obtained polymers photoinduced by this NMP² initiator display a distinct polymer network arrangement. This leads to significantly improved mechanical properties, including increased flexibility, up to 3-fold increase in elongation to break, and up to a 2-fold enhancement in overall toughness.

### Polymerization Methods

One embodiment provides a method to polymerize a polymerizable compound or mixture of two or more different polymerizable compounds, comprising contacting the polymerizable compound or the mixture of two or more different polymerizable compounds with a photopolymerization initiator as described herein or a photopolymerization initiators of formula I wherein:

W is an aryl, wherein the aryl is optionally substituted with one or more groups independently selected from the group consisting of halo, —OH, (C₁-C₄)alkyl, and —C(═O)Ph wherein the —C(═O)Ph is optionally substituted with halo, —OH, or (C₁-C₄)alkyl;
each R¹ is independently (C₁-C₄)alkyl; and
each R² is independently (C₁-C₆)alkyl.

In one embodiment the photopolymerization initiator is less than 1 wt %.

In one embodiment the photopolymerization initiator is less than about 0.9 wt %.

In one embodiment the photopolymerization initiator is less than about 0.85 wt %.

In one embodiment the photopolymerization initiator is less than about 0.8 wt %.

As used herein the wt % refers to the wt % of a mixture, such as the mixture comprising the photopolymerization initiator and the polymerizable compound(s).

In one embodiment the photopolymerization initiator and the polymerizable compound(s) are exposed to UV light.

In one embodiment the polymerization method does not include the polymerization of t-butyl acrylate by NMP² 1.

The invention will now be illustrated by the following non-limiting Examples.

### Example 1

### Experimental Section

Materials: To synthesize the chromophore containing alkoxyamine based NMP² initiators, 4-Aminobenzophenone, 1-nitroso-2-naphthol (97%), 2-aminoanthracene (96%), 2-bromo-2-methyl-propionic acid methyl ester (99%), N,N,N′,N′,N″-pentamethyl-diethylenetriamine (PMDETA, 99%), copper(I) bromide (CuBr, 98%), copper (Cu(0), 99%), benzene (99.8%), diethyl ether (≥99.0%) and hexyl acrylate (HA, 98%) were purchased from Sigma Aldrich and used as received. Hydrogen peroxide (H₂O₂, 30%), magnesium sulfate anhydrous (powder/certified), and potassium hydroxide concentrate (KOH) were purchased from Fisher Scientific and used as received. 2,2-dimethoxy-2-phenylacetophenone (DMPA, Ciba Specialty Chemicals), and phenylbis(2,4,6-trimethylbenzoyl)phosphine oxide (BAPO, Ciba Specialty Chemicals) were used to initiate the radical polymerization.

Methods: A UV/Vis spectrometer (Varian Cary 50 bio) was used to obtain the optical properties of NMP² initiators. An acetonitrile diluted sample was positioned so that a UV curing light source could transmit through the material. The absorbance spectrum was obtained by measuring the intensity changes of light that passed through a sample with respect to the intensity of light through a reference (neat acetonitrile solvent). The molar absorptivity (ε) was calculated using Beer-Lambert's law as shown in equation 1:

$$A = ε \times c \times l \qquad ( 1 )$$

where A is the absorbance, ε is the molar absorptivity, and is the path length. During the UV irradiation, the dynamic changes in spectrum and/or a specific absorption peak of a sample was monitored in real time to determine the photo-decay property for each NMP² initiator. The UV irradiation was provided through a high-pressure mercury lamp (Omnicure S1500 spot cure system equipped with a 320-500 nm bandpass filter) at 20 mW/cm².

To measure the polymerization kinetics, the real-time conversion was monitored using a Thermo Nicolet Nexus 670 real-time Fourier transform infrared spectrometer (RT-FTIR) at ambient temperature (25° C.). A small amount of monomer solution was placed between two NaCl salt plates and subsequently illuminated at room temperature with a high-pressure mercury arc lamp (Omnicure S1500 spot cure system equipped with a 320-500 nm bandpass filter) at 20 mW/cm². During UV illumination, the acrylate conversion was measured by following the decrease of the acrylate peak at 810 cm⁻¹ (C═C twisting). Real time monomer conversion was calculated by Equation 2:(30)

$$Conversion ( \% ) = \frac{{h}_{0} - {h}_{t}}{{h}_{0}} \times 100 \qquad ( 2 )$$

where h₀ is the initial height of the C═C absorption twisting peak before photocuring and hₜ is the peak height at any time during photocuring (31).

To measure the number average molecular weight and polydispersity index (PDI) of NMP² initiated hexyl acrylate system, a size exclusion chromatography (SEC) attached with a refractive index detector (Shimadzu RID10A) was employed. A sample (30-40 mol/ml) was pre-dissolved into tetrahydrofuran (THF) and injected through a PLgel 5 μm Mixed-D column for experimental analysis. A series of standard styrene samples were used for calibration. To synthesize the alkoxyamine-based NMP² initiator, the nitroso-containing chromophores were reacted with PMDETA to derive designed alkoxyamine-based NMP² initiators (32). (4-Nitrosophenyl)(phenyl)methanone and 2-nitrosoanthracene were synthesized following a previously published procedure to oxidize the amino group of aniline to the nitroso group (33). The compounds were synthesized as described below:

Synthesis of NMP² 1 precursor: (4-nitrosophenyl)(phenyl)methanone: A solution of 1.97 g (0.01 mol) 2-aminobenzophenone in 3 mL of methanol was prepared. Three mL of methanol (0.04 mol, 4 equal moles of 2-aminobenzophenone) and 4.5 mL of distilled water were added to the mixture and stirred at room temperature. When fine crystals had precipitated on the bottom, 0.144 g (0.001 mol) molybdenum trioxide (MoO₃) and 1 mL (0.001 mol) of aqueous KOH solution were added, and the solution was stirred at 25° C. for 4 days. Fifteen mL of distilled H₂O were added, and the (4-nitrosophenyl)(phenyl)methanone precipitate was filtered and washed with H₂O (2×10 mL) and cold methanol (5 mL), and then dried. The final product obtained (4-nitrosophenyl)(phenyl)methanone was a yellow-brown color.

Synthesis of NMP² 3 precursor: 2-nitrosoanthracene: A solution of 1.93 g (0.01 mol) 4-aminoanthracene in 3 mL of methanol was prepared. Three mL of methanol (0.04 mol, 4 equal moles of 2-aminobenzophenone) and 4.5 mL of distilled water were added to the mixture and stirred at room temperature. When fine crystals had precipitated on the bottom, 0.144 g (0.001 mol) molybdenum trioxide (MoO₃) and 1 mL (0.001 mol) of aqueous KOH solution were added, and the solution was stirred at 25° C. for 4 days. Fifteen mL of distilled H₂O were added, and the 2-nitrosoanthracene precipitate was filtered and washed with H₂O (2×10 mL) and cold methanol (5 mL), and then dried. The final obtained 2-nitrosoanthracene was a yellow-brown color.

Synthesis of alkoxyamine-based NMP² 1: methyl 2-((4-benzoylphenyl)((1methoxy-2-methyl-1-oxopropan-2-yl)oxy)amino)-2-methyl propanoate: Under inert conditions, two solutions were prepared. Solution A contained 1.0 g (0.0047 mol) 4-nitrosobenzophenone and 2.57 g (0.014 mol) 2-Bromo-2-methyl-propionic acid methyl ester in 20 mL benzene, and solution B contained 0.90 g (0.014 mol) Cu(0), 2.03 g (0.014 mol) CuBr, and 4.92 g (0.028 mol) PMDETA in 20 mL benzene. Then, solution A was added to B under the inert atmosphere and stirred at room temperature. After 24 hours, the mixture solution was filtered through Celite 545, and then ethyl ether was added to dilute the filtered solution. Distilled water was added until clear phase separation was observed. The organic phase was slowly separated and collected, then dried with a small amount of anhydrous MgSO₄. A rotary evaporator was used to remove all the solvent at room temperature. 1.3 g (1.57 mmol) of NMP² 1 with a yield of 66% was obtained as a red, oil-like liquid. ¹H NMR (CDCl₃, 300.13 MHz, δ): 1.24-1.56 (m, 12H), 3.36 (s, 3H), 3.66 (s, 3H), 6.68-6.72 (d, 2H), 7.38-7.76 (m, 6H).

Synthesis of alkoxyamine-based NMP² 2: methyl 2-((1-hydroxynaphthalene-2-yl)((1-methoxy-2-methyl-1-oxopropan-2-yl)oxy)amino)-2-methyl propanoate: Under inert conditions, two solutions were prepared. Solution A contained 0.81 g (0.0047 mol) 1-nitroso-2-naphthol and 2.57 g (0.014 mol) 2-Bromo-2-methyl-propionic acid methyl ester in 20 mL benzene, and solution B contained 0.90 g (0.014 mol) Cu(0), 2.03 g (0.014 mol) CuBr, and 4.92 g (0.028 mol) PMDETA in 20 mL benzene. Then, solution A was added to B under the inert atmosphere and stirred at room temperature. After 24 hours, the mixture solution was filtered through Celite 545, and then ethyl ether was added to dilute the filtered solution. Distilled water was added until clear phase separation was observed. The organic phase was slowly separated and collected, then dried with a small amount of anhydrous MgSO₄. A rotary evaporator was used to remove all the solvent at room temperature.0.9 g (2.40 mmol) of NMP² 2 with a yield of 51% was obtained as a brown oil-like liquid. ¹H NMR (C₆D6, 300.13 MHz, δ): 1.10-1.23 (m, 12H), 3.29 (s, 6H), 6.17 (s, 1H), 7.50-7.60 (m, 3H), 8.35-8.37 (d, 3H).

Synthesis of alkoxyamine-based NMP² 3: methyl 2-(anthracene-2-yl((1-methoxy-2-methyl-1-oxopropan-2-yl)oxy)amino)-2-methyl propanoate: Under inert conditions, two solutions were prepared. Solution A contained 0.97 g (0.0047 mol) 4-nitrosoanthracene and 2.57 g (0.014 mol) 2-Bromo-2-methyl-propionic acid methyl ester in 20 mL benzene, and solution B contained 0.90 g (0.014 mol) Cu(0), 2.03 g (0.014 mol) CuBr, and 4.92 g (0.028 mol) PMDETA in 20 mL benzene. Then, solution A was added to B under the inert atmosphere and stirred at room temperature. After 24 hours, the mixture solution was filtered through Celite 545, and then ethyl ether was added to dilute the filtered solution. Distilled water was added until clear phase separation was observed. The organic phase was slowly separated and collected, then dried with a small amount of anhydrous MgSO₄. A rotary evaporator was used to remove all the solvent at room temperature. 0.83 g (4.19 mmol) of NMP² 3 with a yield of 43% was obtained as a pale brown oil-like liquid. ¹H NMR (CDCl₃, 300.13 MHz, δ): 1.22 (s, 6H), 1.53 (s, 6H), 3.64 (s, 6 H), 6.98 (s, 1H), 7.35 (s, 1H), 7.45-7.54 (m, 3H), 7.94-8.01 (q, 2H), 8.39 (s, 1H), 8.46 (s 1H).

### Results and Discussion

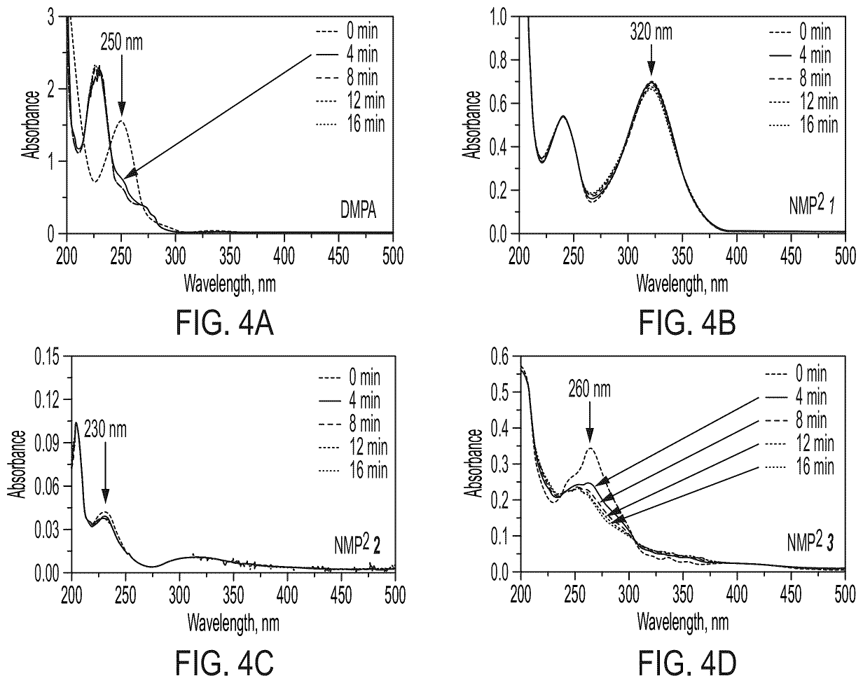

Ultraviolet-visible spectroscopy (UV-vis) was utilized to investigate the light absorption properties of alkoxyamine-based NMP² initiators. FIG. 3 shows the UV-vis absorption spectrum of synthesized initiators 1, 2, and 3. Two conventional photoinitiators, DMPA and BAPO, are also tested and used as the controls. NMP² initiators show UV light absorbance from 200 to 400 nm with the maximum absorption observed at 320 nm for 1, 230 nm for 2, and 260 nm for 3 (FIG. 3). Table 1 shows the molar absorptivity (ε) of NMP² initiators at 365 nm and 405 nm, traditional photocuring wavelengths. At 365 nm, NMP² 1 and 3 exhibit ε values which are about 4-fold and 2-fold higher than DMPA, while a lower E is observed from NMP² 2 initiator. These light absorbance properties at 200-400 nm indicate the potential of using NMP² initiator as a photoinitiator under UV light irradiation. In addition, the light absorption range also varies depending on the chromophore structures of NMP² initiators. When comparing the molar absorptivity at the higher wavelength of 405 nm, neither DMPA nor NMP² 1 initiator shows significant light absorbance. On the other hand, NMP² initiators 2 and 3 exhibit an expanded absorption wavelength range. In particular, the NMP² 3 initiator not only has a higher ε value at 405 nm (ca. 327 M⁻¹ cm⁻¹) but also shows extended absorbance as high as 450 nm. This red-shift of absorption in UV and visible regions can be explained by electron transitions and varied mobility of w electrons in different conjugated systems. In general, w electrons that come from a relatively large conjugated system are more susceptible to lower-energy photons (34). This phenomenon can be explained by the photon energy (E) function which is defined by E=hv/λ, where h is Planck's constant, v is the speed of light in the vacuum, and λ is the wavelength of photon. According to this photon energy equation, photons emitted from longer wavelengths have lower energy. Therefore, as the size of the conjugated system increases the absorption wavelength tends to shift toward the long-wavelength absorption region. Since anthracene-derived NMP² 3 has the largest conjugated system (consisting of three fused benzene rings), the highest wavelength absorption range is observed from the NMP² 3, followed by NMP² 2 (two fused benzene rings) and NMP² 1 (containing two separated benzene rings).

TABLE 1
Measured maximum absorption wavelength (λ), and molar absorptivity
((ε) at 365 nm and 405 nm) of synthesized NMP² initiators
1, 2, and 3. The corresponding parameters of DMPA and BAPO were
also measured. All samples were prepared by using acetonitrile
as the solvent and absorption observed at room temperature

| | λ (nm) at | ε at 365 nm | ε at 405 nm |
|---|---|---|---|
| Initiator | maximum ε | (M⁻¹ cm⁻¹) | (M⁻¹ cm⁻¹) |
| DMPA | 250 | 121 | 0 |
| BAPO | 250 | 986 | 669 |
| NMP² 1 | 320 | 415 | 0 |
| NMP² 2 | 230 | 12 | 3 |
| NMP² 3 | 260 | 215 | 327 |

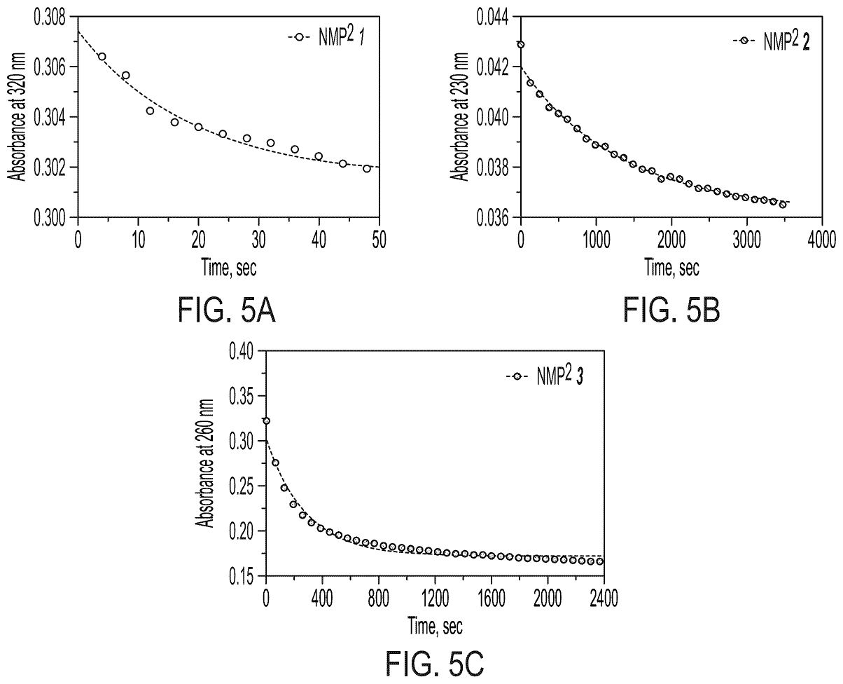

Cleavage of NMP² photoinitiators: In addition to measuring the effective light absorption range of NMP² photoinitiators, UV-Vis spectroscopy was also used to monitor the photolysis behavior of NMP² photoinitiators. As shown in scheme 1, upon irradiation, the C—ON bond of the alkoxyamine compound (in the NMP² initiator) is cleaved to generate a carbon-centered radical together with a nitroxide radical. Typically, the photoproducts (carbon-centered radical and nitroxide radicals produced through dissociation) have different molecular structures compared to the parent materials and result in altered light absorption properties in the ultraviolet and visible light regions. These spectrum changes of NMP² initiators were monitored under light irradiation in inert conditions. As shown in FIG. 4, after irradiating for 20 minutes, the DMPA sample shows absorbance increase at 225 nm and a decrease at 250 nm, indicating significant photobleaching during DMPA decomposition. On the other hand, the differences in the spectrum for NMP² samples before and after light irradiation are relatively small, suggesting limited photobleaching when compared to the control (DMPA). This phenomenon can be explained by the small absorbance differences between NMP² initiators and their corresponding nitroxide radicals (photoproducts) after cleavage. The chromophore compound, which is mainly responsible for light absorption, is not destroyed or changed during photocleavage and remains attached to the formed nitroxide radicals. Thus, limited spectrum changes are observed for NMP² initiators before and after photocleavage.

Scheme 1. The decomposition of the C—ON bond of NMP² initiators forming a carbon-centered radical and a nitroxide radical.

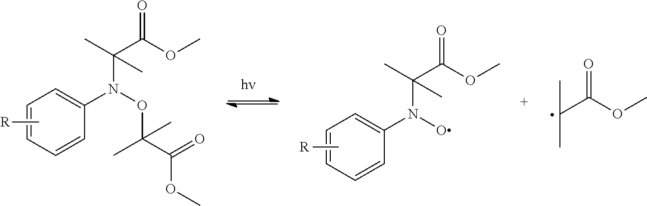

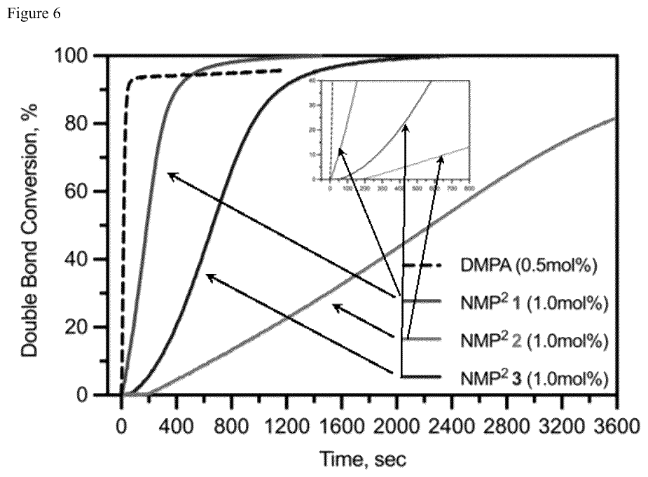

Although the absorption change of NMP² initiators are much smaller than DMPA samples, these small absorbance differences still show evidence of photobleaching. To further understand the photobleaching behavior of synthesized NMP² initiators, an appropriate absorption peak is selected for each NMP² initiator to measure absorption changes under UV light irradiation. FIG. 5 shows the decay of the absorbance peak at 320 nm, 230 nm, and 260 nm for NMP² initiators 1, 2, and 3 in solvent, respectively. By fitting the absorbance changes to an exponential decay curve, the photo-decay constant (k_{decay}) and the half-life (t_{1/2}) of each initiator is calculated and tabulated in Table 2. Under light irradiation, initiator 1 exhibits the highest k_{decay} among NMP² initiators, indicating faster C—ON bond cleavage reaction to generate carbon-centered radicals and corresponding nitroxide-based persistent radicals. Initiator 3 shows a relatively low k_{decay} when compared to 1. The slowest bond cleavage/decomposition process is exhibited by initiator 2 in which the is an order of magnitude less than that of initiators 1 and 3. When comparing the t_{1/2} for those NMP² initiators, the longest t_{1/2} is observed from the NMP² 2 initiator (˜920s), then followed by 3 (˜190s) and 1 (˜14s). These different k_{decay} and t_{1/2} between NMP² initiators suggest the tunable photobleaching properties in NMP² initiators which can, in part, be explained in terms of different molar absorbtivities. The highest molar absorptivity of NMP² 1 (FIG. 2) at initiating wavelengths provides potentially higher energy transfer efficiency from the light-absorbing chromophore compound to the alkoxyamine to break the C—ON bond, allowing a rapid generation of primary radicals. Initiator 2, on the other hand, exhibits the lowest wavelength absorption which is expected to take longer for C—ON bond decomposition and show low constant k_{decay}.

TABLE 2
The absorbance decay constant (k_{decay}) and half-life time
(t_{1/2}) for NMP² initiator 1 at peak of 320 nm, 2 at 230 nm, and
3 at 260 nm. All initiators were dissolved in acetonitrile (c =
1.20 × 10⁻⁴ M) and absorbance measured at room temperature

| NMP2 initiator | 1 | 2 | 3 |
|---|---|---|---|
| k_{decay} (s⁻¹) | 5.2 × 10⁻³ | 7.5 × 10⁻⁴ | 3.6 × 10⁻³ |
| t_{1/2} (s) | 14 | 920 | 190 |

### NMP² Initiated Homopolymerization

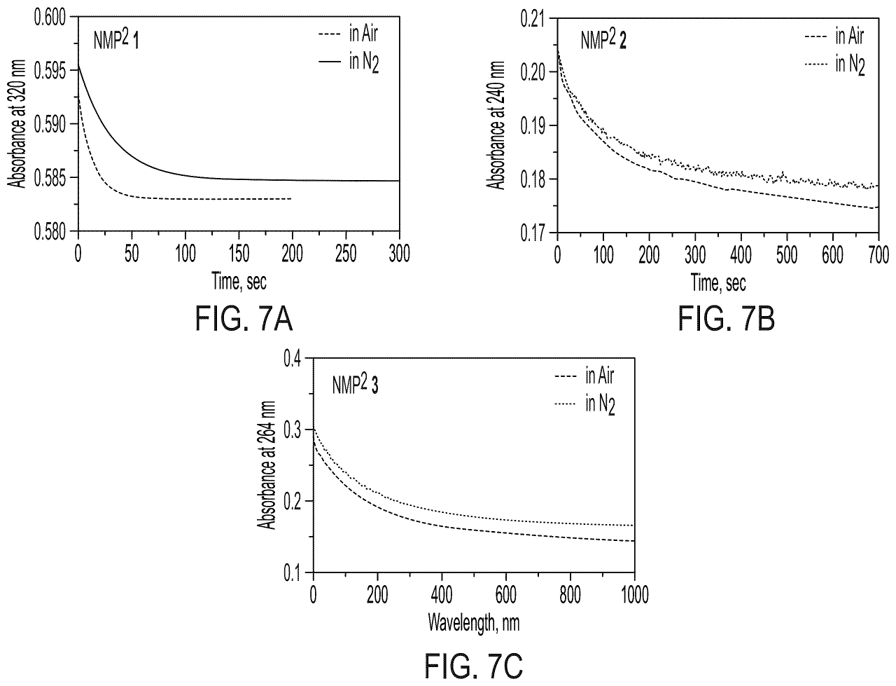

In order to investigate the photocuring capacity of NMP² initiators, the monovinyl hexyl-acrylate was photoinitiated with NMP² initiator under UV light irradiation (320-500 nm filter, 20 mW/cm²). FIG. 6 presents the double bond conversion as a function of time for formulations initiated with NMP² 1, 2, and 3. DMPA-containing formulations were examined. Ideally, DMPA photocleavages two propagating radicals, whereas NMP² initiators produce only one primary radical to initiate polymerization. In order to keep the overall primary radicals generated/produced from different initiator systems similar, the concentration of NMP² initiator is fixed at 1.0 mol % while 0.5 mol % of DMPA initiator is added to the control formulation. As shown in FIG. 5, DMPA induces a high final monomer conversion of around 96%. When using NMP² initiators to initiate the model system, formulations containing 1 and 3 exhibit high final conversions of almost 100% while the formulation containing initiator 2 shows a reduced conversions of around 80%. When comparing the photopolymerization rate indicated by the slope of the conversion curve, NMP² formulations exhibit significantly slower reaction rates compared to the control, but at different rates. Initiator 1-containing hexyl acrylate polymerization exhibits the fastest reaction rate, taking approximately 800 seconds to achieve the final conversion. NMP² 3 initiated polymerization exhibits a slower polymerization rate than 1 and approaches the final conversion within 2000 seconds. The slowest polymerization rate is observed from 2 which needs about 3600 seconds to achieve final conversion. These altered polymerization rates observed from NMP²-initiated photopolymerization are expected due to the inherently reversible nature of NMP processes. For an ideal reversible deactivation mechanism, propagating radicals combine with (macro)nitroxide radicals to temporarily terminate chain propagation and form the poly-NMP² intermediates as dormant chains. Then poly-NMP² intermediates undergo further dissociation to enable reinitiation with monomers or recombine again with other nitroxide radicals. As a result, a series of reversible termination and reinitiation cycles are established, leading to reduced polymerization rates.

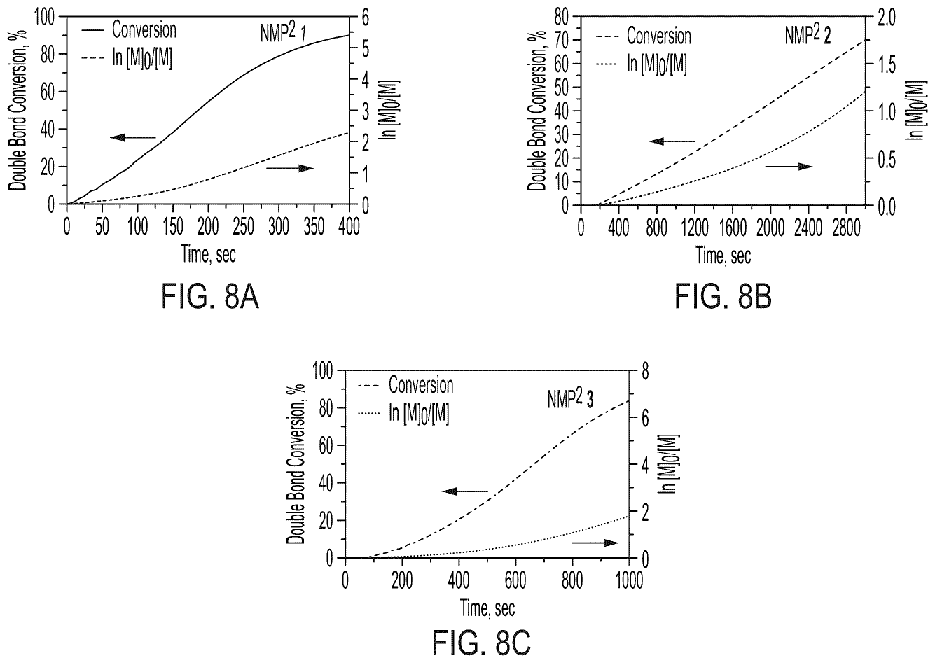

In order to investigate the reversibility of these NMP² initiators during the free radical photopolymerization process, the lifetime of alkoxyamine functionality in NMP² initiators is studied through the additional photo-decay test under ambient conditions (FIG. 7). In this situation, oxygen acts as a free radical scavager to quench/deactivate propagating radicals and prevents the recombination reaction after C—ON bond cleavage, leading to a rapid decrease rate in NMP² initiator photolysis rate. On the other hand, under an inert atmosphere, a slow photolysis rate is observed from NMP² initiators in the same timeframe, suggesting that the recombination reaction between nitroxide radicals and propagation radicals is more favored, leading to a prolonged lifetime of propagating radicals and the “living” characteristic of NMP² initiators (35). In addition, according to the 3-stage description of the NMP kinetics, such long C—ON bond decomposition also ensures higher control over propagating chains which further prevents self-termination (35).

This prolonged lifetime of propagating radicals and NMP²-based controlled radical polymerization are also supported by the first-order behavior in NMP²-initiated polymerization. During the chain propagation, by assuming the long-chain approximation (n>>1) and the propagating rate constant is independent of radical chain length, the propagation rate (RP) can be expressed as shown in equation 3,

$${R}_{p} = \frac{d [ M ]}{dt} = {k}_{p} [ {M}_{n} \cdot ] [ M ] \qquad ( 3 )$$

where [Mₙ^{•}] is the propagating chains, M is the monomer, and kₚ is the propagation constant. Integrating equation 3 derives the relationship between 1n([M]₀/[M]) and curing time, as shown in equation 4.

$$ln \frac{{[ M ]}_{0}}{[ M ]} = {k}_{p} [ {M}_{n} \cdot ] t \qquad ( 4 )$$

For an ideal controlled radical polymerization, the reversible deactivation and activation mechanism will lead to a steady [Mₙ^{•}] which can be indicated by the constant slope in equation 4. As shown in FIG. 8, a linear relationship between the 1n([M]₀/[M]) and time is observed. This linear relationship in the 1n([M]₀/[M]) curve indicates a steady concentration of propagating radicals during the chain propagation process,[36] supports that NMP² based photopolymerization enables controlled radical chain propagation due to the mediation of nitroxide radicals which decomposed from NMP² initiators. In addition, different NMP² initiators show different time ranges for the linear 1n([M]₀/[M]) relationship. Initiator 1 formulations (FIG. 8A) exhibit a linear 1n([M]₀/[M]) for the first 400 seconds by which 90% of conversion is obtained. Systems with 3 (FIG. 8C) show approximately 1000 seconds of a linear 1n([M]₀/[M]) profile that leads to 80% conversion. In the case of formulation initiated by 2 (FIG. 8B), the linear region in 1n([M]₀/[M]) continues for 3000s up to 70% conversion.

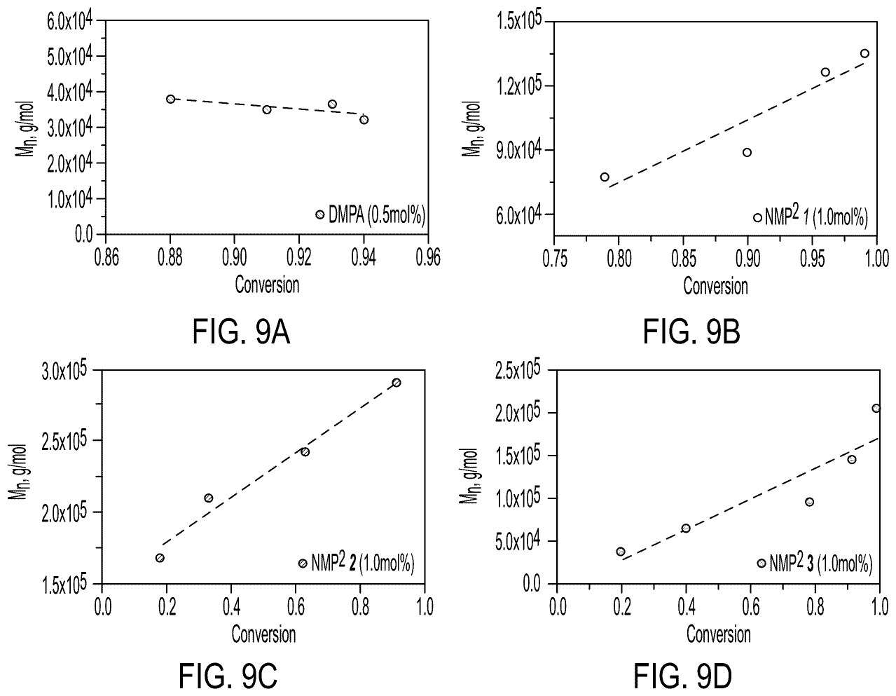

Due to the observed controlled radical polymerization with NMP² initiator-initiated polymerization, it is reasonable to believe different polymer chains are created. The polymer chain evolution behavior was also investigated by polymerizing hexyl acrylate with NMP² initiator. The molecular weight and polydispersity index (PDI) of the model polymer at different conversions were examined by GPC. The number average molecular weight (Mₙ) of poly(hexyl acrylate) photoinitiated with DMPA or NMP² initiators are plotted as a function of monomer conversion in FIG. 9. The DMPA-initiated system shows a typical free radical/chain-growth polymerization manner in which the molecular weight is independent of monomer conversion or curing time (FIG. 9A). However, when substituting DMPA with NMP² photoinitiators, a linear relationship between molecular weight and conversion is observed (FIGS. 9B, 9C, and 9D), indicating a controlled polymer chain growth behavior based on the reversible termination and reinitiation process of persistent (macro)nitroxide radicals. Additionally, molecular weight and PDI for the final linear polymer (poly(hexyl acrylate)) are also measured and listed in Table 3. The NMP² initiator formulations have significantly higher final molecular weight. NMP² 1-containing formulation achieves an Mₙ of 134 KDa, which is 4 times higher than that of the DMPA system. Samples photopolymerized with initiator 3 exhibit further increased molecular weight, close to 200 KDa. The largest molecular weight of poly(hexyl acrylate) is observed from the initiator 2-initiated system, showing a 9-fold higher molecular weight. Samples initiated with NMP² 1, 2, and 3 initiators show PDIs of 2.2, 1.4, and 2.0, respectively. NMP² initiator 2 shows the greatest control in polymerizing hexyl acrylate. Although NMP² initiators 1- and 3- initiated systems do not show a PDI equal to or less than 1.5, the obtained PDI values are still significantly reduced compared to the DMPA formulation, suggesting a partially controlled radical polymerization.

These different PDIs also reflect various reactive efficiencies among NMP² initiators. In this situation, the naphthol-based nitroxide radicals have the longest lifetime of nitroxide radicals (FIG. 8). As a result, the naphthol-based nitroxide radicals from 2 provide a high level of mediation in chain propagating and result in a slow polymerization rate but the highest molecular weight and lowest PDI. For the benzophenone-derived initiator (1), on the other hand, the lifetime of nitroxide radicals is relatively short, leading to the fastest polymerization rate but less control over chain propagation, leading to the lowest molecular weight and broader PDI. The anthracene-derived initiator 3 has an intermediate lifetime, allowing a relatively fast reaction rate similar to 1 and along with more control over chain propagation leading to increased molecular weight and relatively narrower PDI. Based on these results, NMP² initiator 2-initiated polymerization exhibits a high degree of polymer formation control while the initiators 1 and 3 show partially controlled chain propagation behavior.

TABLE 3
The molecular weight and polydispersity of poly(hexyl acrylate) polymerized
with DMPA and NMP² 1, 2, and 3. All samples were photocured under UV irradiation
(320-500 nm band filter with 20 mW/cm²) and were tested using GPC

| Initiator | DMPA | NMP² 1 | NMP² 2 | NMP² 3 |
|---|---|---|---|---|
| Molecular weight | 32000 ± 1000 | 130000 ± 2800 | 290000 ± 3700 | 200000 ± 3600 |
| (g/mol) | | | | |
| Polydispersity index | 2.74 ± 0.06 | 2.20 ± 0.05 | 1.40 ± 0.03 | 2.10 ± 0.01 |
| (PDI) | | | | |

### Conclusion

Various alkoxyamin-based NMP² initiators were synthesized. These initiators enable different levels of control through the reversible deactivation and activation of propagating radicals. NMP² initiators show light absorption in the ultraviolet regions with different photodissociation properties. These NMP² molecules do effectively initiate photopolymerization upon exposure to UV light although at slower reaction rates than conventional systems. This rate decrease indicates that controlled chain propagation processes are occurring. As the nitroxide radicals formed from dissociated NMP² initiators enable reversible termination and fragmentation during the chain propagation process, leading to linear relationship between molecular weight and double bond conversion. The final linear polymers also show up to 9 times higher molecular weight and significantly decreased polydispersity. These alkoxyamine-derived NMP² initiators provide a new means to apply controlled radical polymerization through a nitroxide-mediated polymerization mechanism including significantly different polymerization behavior and regulating polymer chain length and distribution.

### Example 2

### Experimental Section

Materials: The acrylate oligomer mixture (Ebecryl 605/40, Allnex) composited of 60 wt % bisphenol A epoxy diacrylate and 40 wt % tri(propylene glycol) diacrylate (TPGDA) was used as a model cross-linked formulation to polymerize with NMP² initiators. 2,2-dimethoxy-2-phenylacetophenone (DMPA, Ciba Specialty Chemicals) photoinitiator (PI) was used in comparison as a conventional initiator. The NMP²-based alkoxyamines 1, 2, and 3 were prepared according to the procedures reported in Chapter 6. For formulation preparation, in order to keep all potential initiating radicals similar, NMP² formulations contain a 1.0 mol % of NMP² initiator while 0.5 mol % of DMPA is added for control systems.

Photorheological properties: Real time photorheological properties of alkoxyamine-based NMP² initiator-initiated acrylate formulations were examined with a Kinexus Ultra+ rheometer (Malvern Panalytical) which is equipped with an 8 mm diameter upper plate and a high-pressure mercury arc lamp (Omnicure S2000 spot cure system, 320-500 nm filter). A constant gap distance of 0.12 mm was set between the upper plate and bottom glass plate which is the thickness of photocured polymer films. To ensure full contact between the upper plate and the bottom plate, about 10 μL of the testing formulation was pipetted onto the bottom glass plate. All samples were tested in a strain-controlled oscillatory mode (0.1% strain rate) at a frequency of 1 Hz under UV irradiation. During the measurement, experimental data were collected every 0.05 seconds.

Fourier Transform Infrared Spectroscopy: A Thermo Nicolet Nexus 670 real-time Fourier transform infrared spectrometer (RT-FTIR) was used to monitor the real-time conversion and reaction rates. For sample preparation, a small amount (0.2-0.3 mg) of sample formulation was placed between two NaCl salt plates and subsequently photocured by a high-pressure mercury arc lamp (Omnicure S1500 spot cure system, 320-500 nm bandpass filter) with 20 mW/cm² light at room temperature (25° C.). During UV illumination, the monomer conversion was measured by monitoring the disappearance of the acrylate absorption peak at 810 cm⁻¹ (C═C twisting) compared to the carbonyl reference peak area at 1730 cm⁻¹.

Dynamic mechanical analysis: Thermo-mechanical properties of the polymers, including glass transition temperature (T_{g}), storage modulus (E′), bending stress, and stress-strain property, were measured by a TA Instruments Q800 dynamic mechanical analyzer (DMA). For thin film sample preparation, sample formulations were placed between two glass slides with 0.12 mm separators and photopolymerized using a high-pressure mercury arc lamp (Omnicure S1500 spot cure system, 320-500 nm bandpass filter). All samples were photocured with a light intensity of 20 mW/cm². Polymer samples were cut into small rectangular sizes with dimensions of 7.50×6.20×0.12 mm (L×W×T). To measure the tan delta response to the temperature for model acrylate polymer networks, the temperature sweep mode with constant strain (0.1%) at a frequency of 1 Hz was selected. The heating rate was fixed at 2° C./min from −20° C. to 140° C. The peak of the tan delta curve was used to determine the glass transition temperature (T_{g}). To characterize the bending behavior of the NMP² initiator-initiated epoxy acrylate polymer film, a single cantilever clamp (span is 17.5 mm) was used. In this test, a rectangular sample with dimensions of 17.50×6.20×0.54 mm (L×W×T) was selected to ensure the span to thickness ratio was 16:1. The bending stress was tested as a function of deflection and the flexural modulus was found from the slope of the linear regions in the bending stress-deflection curves. Tensile properties, such as elongation to break and tensile strength, were evaluated under tensile testing mode with a force rate of 2.0 N/min at 25° C. Tensile modulus was determined by the slope of the initial linear section from the stress-strain curve, and the overall toughness was calculated as the area under stress-strain curves until failure.

### Results and Discussion

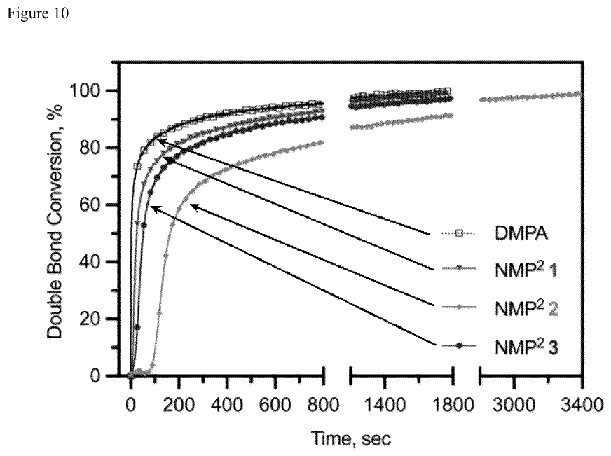

Nitroxide-mediated Photopolymerizayion: In order to investigate the photocuring capacity of NMP² initiators in a cross-linked photocurable system, acrylate oligomer Ebecryl 605/40 was polymerized with NMP²-based alkoxyamine initiators. FIG. 10 presents the double bond conversion as a function of time for formulations photoinitiated with different NMP² initiators 1, 2, and 3. DMPA-initiated formulations were tested as a control. All NMP²-initiated and DMPA formulations exhibit similar high monomer conversions (˜99%). However, the polymerization rate, represented by the slope of the conversion curve, appears to be significantly different in each system. DMPA, with typical free radical initiation, induces a fast polymerization rate while the polymerization rates of NMP² systems are all slower, but to different degrees. When polymerizing with NMP² initiators 1 and 3, a decreased polymerization rate is observed approaching 90% of conversion within 30 minutes. When initiating with initiator 2, the slowest polymerization rate is seen which requires approximately 60 minutes to achieve the same conversion. These changes in the polymerization rates for formulations initiated with NMP² initiators can be explained by the different photo-dissociation properties between NMP² initiators under light irradiation. According to the previous photolysis study in last chapter, initiator 1 shows the fastest photo-decay coefficient followed by 3, thereby producing more primary radicals to propagate with monomers and leading to faster polymerization rates. Initiator 2, on the other hand, has the lowest photo-decay coefficient under the same conditions, resulting in a much slower polymerization rate. In addition, the 2 formulation also shows a conversion inhibition of about 100 seconds in the early stages of polymerization which supports the low photodissociation rate of 2 compared to other NMP² initiators.

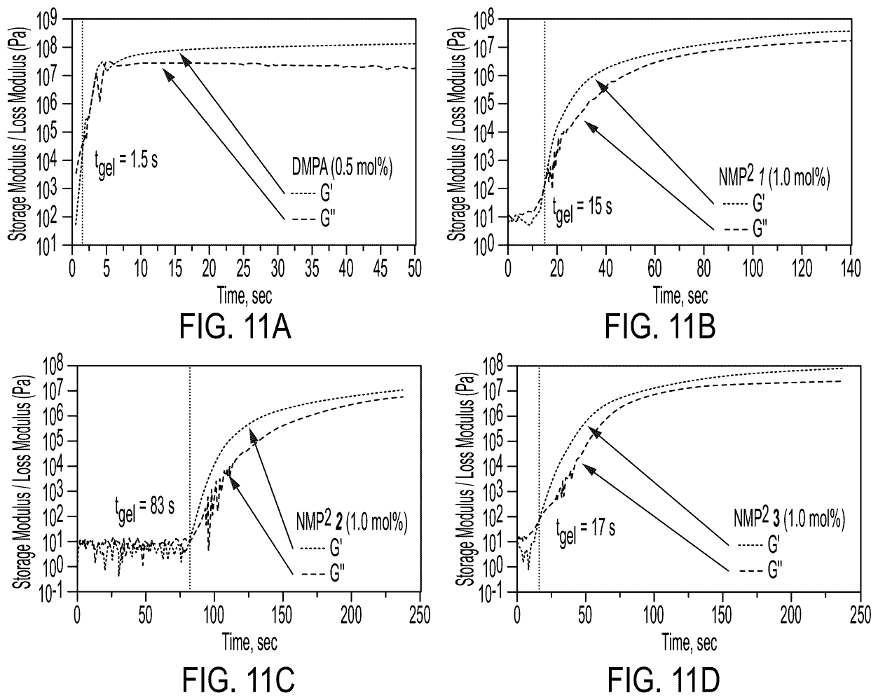

It is well accepted that the controlled radical polymerization leads to delayed gelation in network formations (37-39). Therefore, it is reasonable to believe that the reversible termination mechanism in NMP²-based polymerization may also delay gelation and alter the formation of polymer networks. To determine if these initiators change inherent network evolution and formation, the time to gelation (t_{ge1}) and shrinkage stress during the polymerization using NMP² initiation were evaluated. t_{ge1} is defined as the transition of a cross-linking system from a liquid-like viscous phase to a solid-like elastic phase (40). Often, t_{ge1} is determined by the intersection of the storage modulus (G′) and the loss modulus (G″) during the cure (40,41). FIG. 11 shows the storage and loss moduli as a function of time for model cross-linked formulations initiated with DMPA and NMP² initiators. The DMPA sample exhibits a fast gelation time (t_{ge1}=1.5 s, FIG. 11A) due to its fast polymerization rate and rapid cross-linking. Substituting the DMPA for NMP² initiators, t_{ge1} is significantly delayed to 15, 83, and 17 seconds for initiator 1, 2, and 3 containing formulations, respectively (FIGS. 11A, 111B, and 11C). These delayed gelation times indicate that network evolution is altered when incorporating NMP² initiator into cross-linked systems (42). Although t_{ge1} is measured using a photorheometer using a light intensity of 500 mW/cm², the overall reaction should follow a similar trend. Using high light intensity would generate more free radicals than that induced by low light intensity. But once the propagating radicals are formed, these radicals would either propagate with unreactive monomers/oligomers or be trapped by nitroxide persistent radicals to undergo the reversible deactivation process of the NMP. On the other hand, the increased concentrations of propagating radicals produced from high light intensity may lead to a higher degree of irreversible termination that leads to more limited control over chain growth. In contrast, when low light intensities are utilized for photocuring, a higher degree of reversible deactivation control will mediate chain propagating and network evolution, leading to further delayed gelation in comparison to that initiated under higher light intensity. However, the observed trend of delayed gelation is still valid at high light intensity.

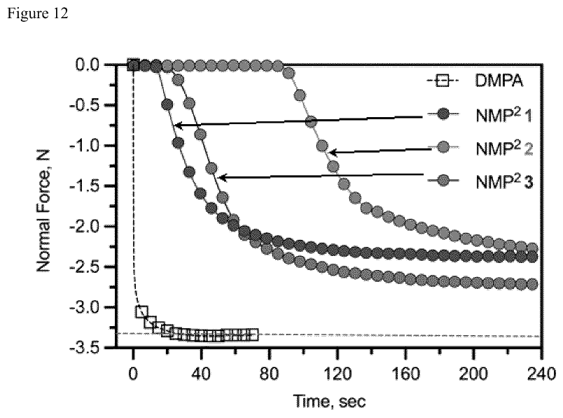

To further understand the polymer network formation photoinitiated by NMP² initiators, the volume shrinkage during polymerization was measured by photorheometry. The shrinkage stress can be determined by the changes in normal force monitored on the oscillating plate of the photorheometer (41, 43, 44). According to FIG. 12, a significant delay in normal force onset time is observed in the NMP²-containing formulations, indicating the longer vitrification time for formulations initiated by NMP² initiators. In addition, when the normal force curve approaches a flat plateau, the polymerization reaction is close to final conversion, and the corresponding normal force indicates the extent of shrinkage stress that occurs during the polymerization. The final normal force of the DMPA formulation is around −3.4 N. However, significant reductions in shrinkage stress are observed from the NMP²-initiated formulations. Normal force drops by 20% in NMP² 3 initiated formulations. In NMP² 1 or 2 containing systems, up to 30% reduction in normal force is observed compared to the control. These results confirm that nitroxide radicals dissociated from NMP² initiators are able to mediate propagating radicals/chains to control the network evolution and mitigate any pre-formed shrinkage stress through viscous flow prior to gelation, leading to significantly delayed gelation and reduced shrinkage stress at final conversions. Again, while the use of high light intensities results in different normal forces at the final conversion state than those using low light intensity, the decreasing trend of the normal force will remain the same by photocuring NMP² initiator containing formulations at lower light intensity.

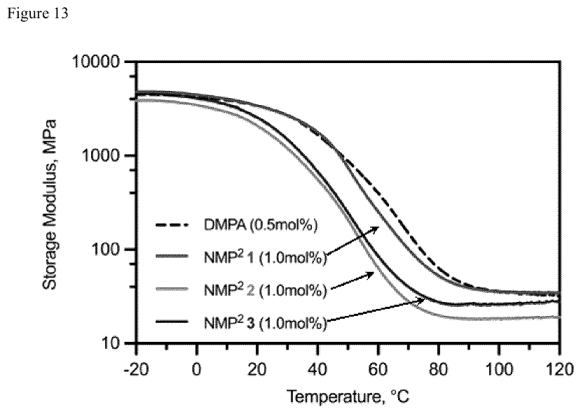

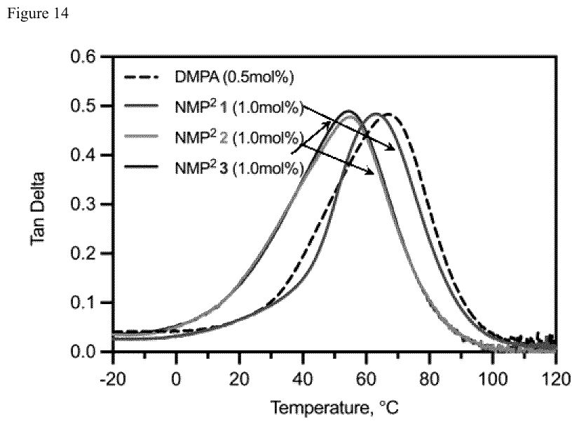

Because of these changes in polymerization kinetics, cross-linking, and shrinkage, different polymer networks are formed by using NMP² initiators implying that thermo-mechanical properties would also be. To this end, the glass transition temperature (T_{g}) and storage modulus (E′), the energy required to induce strains were evaluated with dynamic mechanical analysis. Storage modulus measures the energy required to distort samples (45). As shown in FIG. 13, in rubbery regions, initiator 1 formulations exhibit a comparable storage modulus to DMPA formulations. For initiator 3, the resulting network shows a decreased storage modulus, whereas formulation initiated with 2 exhibits the lowest storage modulus, which is about 40% less than the control. Decreased storage moduli have also been reported from a thermally-induced NMP of butyl methacrylate and ethylene glycol dimethyl acrylate mixtures (46). This phenomenon may likely be due to the formation of lower cross-link density domains within the network after NMP² modification. This hypothesis is supported by the lower T_{g} observed from NMP²-containing networks as shown in FIG. 14 which shows the tan delta curve response to the temperature of model formulations polymerized with different NMP² initiators. The DMPA formulation shows the highest T_{g}. When formulations are initiated with the NMP² initiators, the T_{g} of the resulting polymer network shifts toward lower temperatures, with initiators 1, 2, and 3 at 81° C., 67° C., and 67° C., respectively.

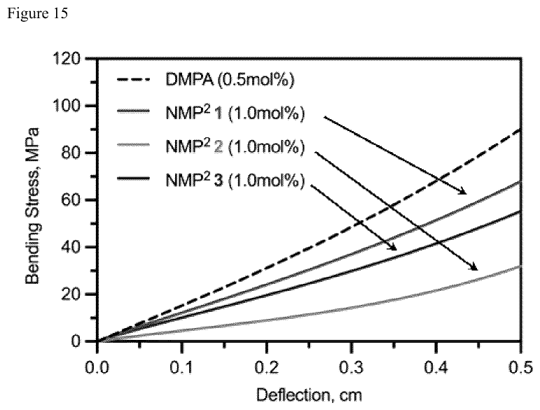

To further investigate network structure changes due to using alkoxyamine-based NMP² initiators, bending stress was examined by performing a single cantilever bending test. FIG. 15 shows the bending test results for the NMP² initiator- and DMPA-initiated polymer films. Bending stress is higher for the DMPA sample than that of NMP²-films, and both increased with deflection. At a low bending deflection of 0.1 cm, the DMPA film requires stress of about 2.3 MPa, while NMP² initiator-containing films exhibit much lower bending stress, 2.1 MPa for initiator 1, 0.8 MPa for initiator 2, and 2.1 MPa for initiator 3. When the deflection continues to high values, bending stresses exerted on the NMP²-modulated films are still lower than that of the DMPA sample, especially for initiator 2 initiated polymer films which show the lowest bending stress (˜3.8 MPa) at a 0.5 cm deflection. On the other hand, almost 4 times the bending stress is required for the DMPA film to achieve the same deflection. These different bending properties of using NMP² initiators suggest the lower flexural modulus in NMP² films, indicating that the corresponding polymer network has a larger free volume/space for chain rotation after NMP² initiator mediation. This increased flexibility further confirms that the NMP²-initiation is capable of generating polymer networks with lower cross-link density.

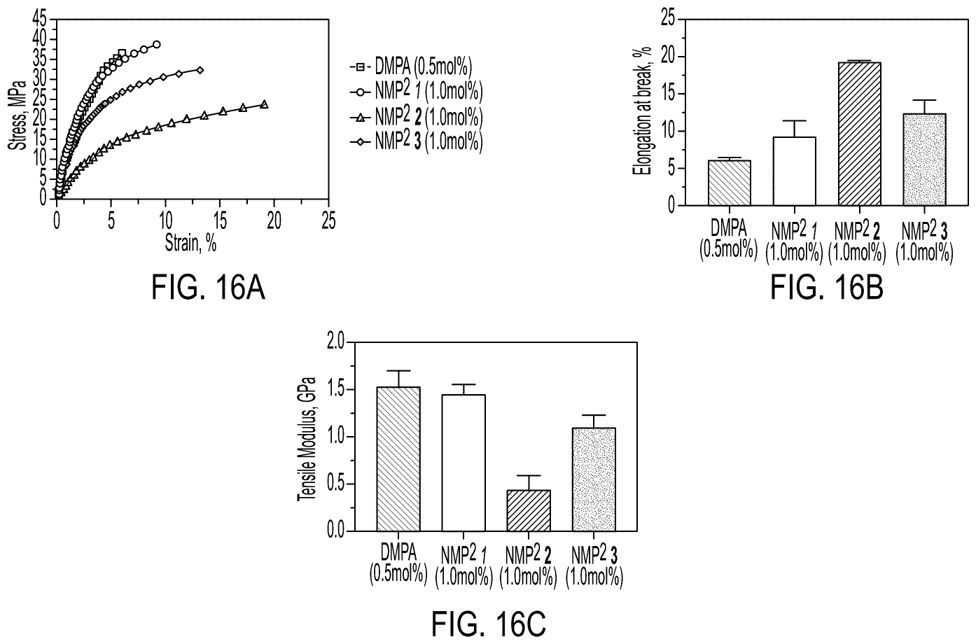

To gain further understanding of the thermo-mechanical properties of NMP²-initiated polymer materials, DMA was used to measure the stress-strain properties, as shown in FIG. 16. The strain to break value, tensile modulus, and tensile strength are tabulated in Table 1. NMP² initiator-initiated polymer films show increased elasticity. Compared to the DMPA system, the elongation at break performance for initiator 1-containing film increases from 6% to 9%. The initiator 3 sample shows further increased ultimate strain to 13%. The highest strain to break value is observed in NMP² 2 modulated film which has 3 times higher elongation at break than the control. However, in terms of film stiffness, a decrease in tensile modulus is observed from NMP²-based photopolymerization. Tensile moduli of samples polymerized with initiators 1 and 3 are 10% and 30% lower than that of the DMPA films while the NMP² 2 samples exhibit the lowest tensile modulus to 0.4 GPa. This reduction trend is also seen from the ultimate tensile stress property. DMPA and NMP² 1 initiated films show similar and largest tensile strength among all samples. The polymer films initiated by 3 exhibit about 16% reduction in ultimate tensile stress, while 2-mediated formulations have the lowest stress, showing only 65% of the controls. This modulation in tensile properties is attributed to the delayed gelation and reduced shrinkage stress within polymer networks. The higher t_{ge1} in NMP² initiator-containing formulations delays the onset of diffusion control in reactive species, leading to much larger primary chains being formed prior to gelation and resulting in the formation of networks that contains higher molecular weight between crosslinks. Due to altered chain propagation and cross-linking, lower shrinkage stress will develop during polymer network evolution. As result, NMP²-initiated polymer films show lower T_{g}, rubbery storage modulus, and tensile modulus but significantly increased elongation to break and enhanced flexibility. In contrast, due to the rapid cross-linking of DMPA-initiated formulation, highly cross-linked dense domains are created in the early stage of polymerization, and higher shrinkage stresses are also generated within networks, resulting in a stiffer but more brittle material.

In addition, the different tensile modulus and strain to break properties of NMP²-films reflect various stabilities between nitroxide radicals that dissociated from NMP² initiators. For example, the long lifetime of naphthol-based nitroxide radicals enables a high extent of reversible deactivation during NMP² 2 initiated propagation, resulting in the lowest network crosslink density which contributes to the low T_{g} and rubbery storage modulus. On the other hand, benzophenone-derived nitroxide radicals cleaved from NMP² 1 initiators are characterized by the shortest lifetime during chain propagation which results in a faster polymerization but lower mediation in polymer network formation, exhibiting lower strain to break values with higher T_{g} and rubbery storage modulus. In the case of anthracene-based NMP² 3, the stability of its corresponding nitroxide radicals is in-between initiators 1 and 2 which allows moderate control in developing polymer networks to yield double elongated polymer films with less reduced tensile modulus while maintaining a relatively fast reaction rate.

TABLE 1
Thermo-mechanical properties for DMPA and NMP² 1, 2, and 3 initiated
polymer (Ebecryl 605/40) films. Samples were photocured under UV light
with 20 mW/cm² using a 320-500 nm bandpass filter

| | DMPA | NMP² 1 | NMP² 2 | NMP² 3 |
|---|---|---|---|---|
| | (0.5 mol %) | (1.0 mol %) | (1.0 mol %) | (1.0 mol %) |
| Strain to break (%) | 6.2 ± 0.3 | 9.4 ± 0.9 | 19.3 ± 0.3 | 13.5 ± 0.9 |
| Tensile modulus (GPa) | 1.5 ± 0.2 | 1.4 ± 0.1 | 0.4 ± 0.1 | 1.1 ± 0.1 |
| Tensile Strength (MPa) | 36.0 ± 3.9 | 38.0 ± 3.9 | 24.0 ± 4.0 | 32.0 ± 2.0 |

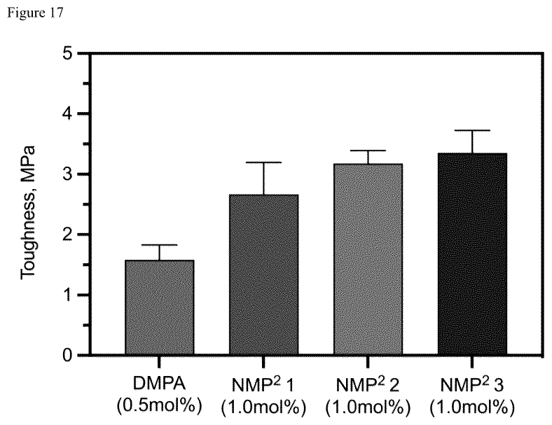

Based on these different thermo-mechanical properties, the overall impact of NMP² initiation on model acrylate materials is also evaluated by measuring the toughness. FIG. 17 shows the toughness of polymer films photoinduced with DMPA and NMP² initiators which is calculated by the area under the stress-strain curves (FIG. 16A). The overall toughness of DMPA sample is around 1.6 MPa. Although polymer films prepared with initiator 1 already show increased toughness to ˜2.7 MPa, incorporating NMP² initiator 2 or 3 results in significantly enhanced toughness (over 3.2 MPa). These enhanced overall toughness in NMP²-initiated polymer materials could be explained again by the inherent nature of NMP² process. A successive reversible termination of propagating radicals/chains mediates chain propagation, delays gelation, and network evolution, leading to the formation of much more homogeneous networks with mitigated shrinkages. NMP² 2-containing formulations, in particular, have greater control over polymer chain mediation to develop lower shrinkage polymer networks, contributing to three-fold higher elongation to break and two-fold enhanced overall toughness in final polymer materials.

### Conclusions

In this research, the effects of using alkoxyamine-based molecules (NMP²) as photoinitiators for cross-linked acrylate formations was examined. Compared to conventional free radical photopolymerization, the incorporation of NMP² initiators decreases reaction rate. However, the dissociated nitroxide radicals enable reversible termination with propagating radicals to mediate chain propagation, leading to delayed gelation time and greater chain relaxation to reduce shrinkage and shrinkage stress in acrylate polymer networks. Tan delta behavior and decreased storage modulus indicate that NMP²-initiator mediated networks have lower cross-link density. On the other hand, NMP²-films show significantly increased flexibility and elongation to break (up to 3-fold), and dramatically enhanced toughness (up to 2-fold). In addition, the different stabilities of nitroxide radicals dissociated from NMP² initiators under light irradiation also affect polymerization kinetics and ultimate thermo-mechanical in polymer materials. Upon light irradiation, highly stable nitroxide radicals in NMP² 2-initiated polymerization provide a high degree of control over chain propagation, leading to the slowest polymerization rate, delayed gelation, and decreased modulus but significantly increased elongation and toughness. In NMP² 1 or 3 initiated polymer films, although benzophenone- and anthracene-based nitroxide radicals have relativity lower stabilities, the corresponding polymer materials still show greater elongation to break and enhanced toughness. Initiator 1 modified film, in particular, not only shows greater elongation at break value (1.5-fold) but also exhibits comparable modulus to the control and faster reaction rates, leading to enhanced overall toughness (1.4-fold). This work shows that NMP²-based controlled radical polymerization is effective to uniquely modifying photopolymer materials.

### DOCUMENTS

1. G. Odian, PRINCIPLES OF POLYMERIZATION Fourth Edition Principles of Polymerization, Fourth Edition, Fourth Edi, John Wiley & Sons, Inc., Hoboken, New Jersey, 2004. www.copyright.com.
2. M. Destarac, Controlled Radical Polymerization: Industrial Stakes, Obstacles and Achievements, Macromol. React. Eng. 4 (2010) 165-179.
3. K. Matyjaszewski, Overview: Fundamentals of Controlled/Living Radical Polymerization, in: Krzysztof Matyjaszewski (Ed.), Control. Radic. Polym., American Chemical Society, 1998: pp. 2-30.
4. K. Matyjaszewski, J. Spanswick, Controlled/living radical polymerization, Mater. Today. 8 (2005) 26-33.
5. H. Fang, A. Guymon, Recent advances to decrease shrinkage stress and enhance mechanical properties in free radical polymerization: a review, Polym. Int. 71 (2021) 596-607.
6. S. Tomoeda, Y. Kitayama, J. Wakamatsu, H. Minami, P. B. Zetterlund, M. Okubo, Nitroxide-Mediated Radical Polymerization in Microemulsion(Microemulsion NMP) ofn-Butyl Acrylate, Macromol. 44 (2011) 5599-5604.
7. A. R. Wang, S. Zhu, Control of the polymer molecular weight in atom transfer radical polymerization with branching/crosslinking, J. Polym. Sci. Part A Polym. Chem. 43 (2005) 5710-5714.
8. N. P. Truong, G. R. Jones, K. G. E. Bradford, D. Konkolewicz, A. Anastasaki, A comparison of RAFT and ATRP methods for controlled radical polymerization, Nat. Rev. Chem. 5 (2021) 859-869.
9. S. Tomoeda, Y. Kitayama, J. Wakamatsu, H. Minami, P. B. Zetterlund, M. Okubo, Nitroxide-Mediated Radical Polymerization in Microemulsion (Microemulsion NMP) of n-Butyl Acrylate, Macromolecules. 44 (2011) 5599-5604.
10. J. Nicolas, Y. Guillaneuf, C. Lefay, D. Bertin, D. Gigmes, B. Charleux, Nitroxide-mediated polymerization, Prog. Polym. Sci. 38 (2013) 63-235.
11. R. B. Grubbs, Nitroxide-mediated radical polymerization: Limitations and versatility, Polym. Rev. 51 (2011) 104-137.
12. G. Delaittre, J. Rieger, B. Charleux, Nitroxide-Mediated Living/Controlled Radical Polymerization of N,N-Diethylacrylamide, Macromolecules. 44 (2011) 462-470.
13. Y. Yagci, S. Jockusch, N. J. Turro, Photoinitiated Polymerization: Advances, Challenges, and Opportunities, Macromolecules. 43 (2010) 6245-6260.
14. S. Telitel, S. Telitel, J. Bosson, A. Spangenberg, J. Lalevee, F. Morlet-Savary, J.-L. Clement, Y. Guillaneuf, D. Gigmes, O. Soppera, Nitroxide Mediated Photopolymerization: A Versatile Tool for the Fabrication of Complex Multilayer Polyfunctional Copolymer Nanostructures, Adv. Mater. Interfaces. 1 (2014) 1400067.
15. H. R. Lamontagne, B. H. Lessard, Nitroxide-Mediated Polymerization: A Versatile Tool for the Engineering of Next Generation Materials, ACS Appl. Polym. Mater. 2 (2020) 5327-5344.
16. W. Xi, H. Peng, A. Aguirre-Soto, C. J. Kloxin, J. W. Stansbury, C. N. Bowman, Spatial and Temporal Control of Thiol-Michael Addition via Photocaged Superbase in Photopatterning and Two-Stage Polymer Networks Formation, Macromolecules. 47 (2014) 6159-6165.
17. E. Rizzardo, D. H. Solomon, On the origins of nitroxide mediated polymerization (NMP) and reversible addition fragmentation chain transfer (RAFT), in: Aust. J. Chem., 2012: pp. 945-969.
18. R. Gonz Alez-Blanco, M. F. Cunningham, E. Sald Ivar-Guerra, High Solids TEMPO-Mediated Radical Semibatch Emulsion Polymerization of Styrene, J. Polym. Sci., Part A Polym. Chem. 54 (2015) 49-62.
19. L. Tebben, A. Studer, Synthetic Methods Nitroxides: Applications in Synthesis and in Polymer Chemistry, Angew. Chem., Int. Ed. 50 (2011) 5034-5068.
20. J. P. Scholte, S. K. Kim, C. L. Lester, C. A. Guymon, Effects of Directed Architecture in Epoxy Functionalized Prepolymers for Photocurable Thin Films, J. Polym. Sci., Part A Polym. Chem. 55 (2016) 144-154.
21. J. C. Scaiano, T. J. Connolly, N. Mohtat, C. N. Pliva, Exploratory study of the quenching of photosensitizers by initiators of free radical “living” polymerisation, Can. J. Chem. 75 (1997) 92-97.
22. S. Hu, J. H. Malpert, X. Yang, D. C. Neckers, Exploring chromophore tethered aminoethers as potential photoinitiators for controlled radical polymerization, Polymer (Guildf). 41(2000) 445-452.
23. A. Goto, J. C. Scaiano, L. Maretti, Photolysis of an alkoxyamine using intramolecular energy transfer from a quinoline antenna-towards photo-induced living radical polymerization, Photochem. Photobiol. Sci. 6 (2007) 833-835.
24. E. Yoshida, Photo-living radical polymerization of methyl methacrylate by a nitroxide mediator, Colloid Polym. Sci. 286 (2008) 1663-1666.
25. E. Yoshida, Photo-living radical polymerization of methyl methacrylate by 2,2,6,6-tetramethylpiperidine-1-oxyl in the presence of a photo-acid generator, Colloid Polym. Sci. 287 (2009) 767-772.
26. E. Yoshida, Nitroxide-mediated photo-living radical polymerization of vinyl acetate, Colloid Polym. Sci. 288 (2010) 73-78.
27. J. Su, X. Liu, J. Hu, Q. You, Y. Cui, Y. Chen, Photo-induced controlled radical polymerization of methyl methacrylate mediated by photosensitive nitroxides, Polym. Int. 64 (2014) 867-874.
28. J. Su, X. Liu, M. Li, T. Zhang, Y. Cui, Stereocontrol of Methyl Methacrylate during Photoinduced Nitroxide-Mediated Polymerization in the Presence of Photosensitive Alkoxyamine, Int. J. Polym. Sci. 2016 (2016) Article ID 6482050.
29. Y. Guillaneuf, D. Bertin, D. Gigmes, D.-L. Versace, J. Lalev, J.-P. Fouassier, Toward Nitroxide-Mediated Photopolymerization, Macromolecules. 43 (2010) 2204-2212.
30. E. Hasa, J. W. Stansbury, C. A. Guymon, Manipulation of crosslinking in photo-induced phase separated polymers to control morphology and thermo-mechanical properties, Polymer (Guildf). 202 (2020) 122699.
31. E. Hasa, J. P. Scholte, J. L. P. Jessop, J. W. Stansbury, C. A. Guymon, Kinetically Controlled Photoinduced Phase Separation for Hybrid Radical/Cationic Systems, Macromolecules. 52 (2019) 2975-2986.
32. A. Defoin, Simple preparation of nitroso benzenes and nitro benzenes by oxidation of anilines with H₂O₂ catalysed with molybdenum salts, Synthesis (Stuttg). (2004) 706-710.
33. E. C. Taylor, C. P. Tseng, J. B. Rampal, Conversion of a Primary Amino Group into a Nitroso Group. Synthesis of Nitroso-Substituted Heterocycles, J. Org. Chem. 47 (1982) 552-555.
34. M. Irfan, K. Xie, A. Hou, Effect of Reactive Dye Structures and Substituents on Cellulose Fabric Dyeing, Fibers Polym. 21 (2020) 2018-2023.
35. G. Audran, E. G. Bagryanskaya, S. R. A. Marque, P. Postnikov, New Variants of Nitroxide Mediated Polymerization, Polymers (Basel). 12 (2020) 1481.
36. R. Henkel, P. Vana, R. Henkel, P. Vana, The Influence of RAFT on the Microstructure and the Mechanical Properties of Photopolymerized Poly(butyl acrylate) Networks, Macromol. Chem. Phys. 215 (2013) 182-189.
37. H. Fang, A. Guymon, Recent advances to decrease shrinkage stress and enhance mechanical properties in free radical polymerization: a review, Polym. Int. 71 (2021) 596-607.
38. W. Wang, Y. Zheng, E. Roberts, C. J. Duxbury, L. Ding, D. J. Irvine, S. M. Howdle, Controlling Chain Growth: A New Strategy to Hyperbranched Materials, Macromolecules. 40 (2007) 7184-7194.
39. M. L. Koh, D. Konkolewicz, S. Ebastien Perrier, A Simple Route to Functional Highly Branched Structures: RAFT Homopolymerization of Divinylbenzene, Macromolecules. 44 (2011) 2715-2724.
40. C. S. Pfeifer, N. D. Wilson, Z. R. Shelton, J. W. Stansbury, Delayed gelation through chain-transfer reactions: Mechanism for stress reduction In methacrylate networks, Polymer (Guildf). 52 (2011) 3295-3303.
41. L. A. Ruppitsch, G. Peer, K. Ehrmann, T. Koch, R. Liska, Photopolymerization of difunctional cyclopolymerizable monomers with low shrinkage behavior, J. Polym. Sci. 59 (2021) 519-531.
42. F. Sun, M. Lin, Z. Dong, D. Zhu, S. L. Wang, J. Yang, Effect of Composition of HPAM/Chromium(III) Acetate Gels on Delayed Gelation Time, J. Dispers. Sci. Technol. 37 (2016) 753-759.
43. C. Gorsche, R. Harikrishna, S. Baudis, P. Knaack, B. Husar, J. Laeuger, H. Hoffmann, R. Liska, Real Time-NIR/MIR-Photorheology: A Versatile Tool for the in Situ Characterization of Photopolymerization Reactions, ACS Anal. Chem. 89 (2017) 4958-4968.
44. G. Peer, P. Dorfinger, T. Koch, J. Rgen Stampfl, C. Gorsche, R. Liska, Photopolymerization of Cyclopolymerizable Monomers and Their Application in Hot Lithography, Macromolecules. 51 (2018) 9344-9353.
45. V. S. Kulkarni, C. Shaw, Rheological Studies, in: V. S. Kulkarni, C. Shaw (Eds.), Essent. Chem. Formul. Semisolid Liq. Dosages, Academic Press, 2016: pp. 145-182.
46. E. Mehravar, A. Agirre, N. Ballard, S. van Es, A. Arbe, J. R. Leiza, M. Asua, K. Aplikatua Saila, K. Zientzien Fakultatea, Insights into the Network Structure of Cross-Linked Polymers Synthesized via Miniemulsion Nitroxide-Mediated Radical Polymerization, Macromolecules. 51 (2018) 9740-9748.

All publications, patents, and patent documents are incorporated by reference herein, as though individually incorporated by reference, including the Thesis titled “Directed Network Structure Through Controlled Radical Photopolymerization” authored by Huang Fang. The invention has been described with reference to various specific and preferred embodiments and techniques. However, it should be understood that many variations and modifications may be made while remaining within the spirit and scope of the invention.

What is claimed is:

1. A photopolymerization initiator of formula I: 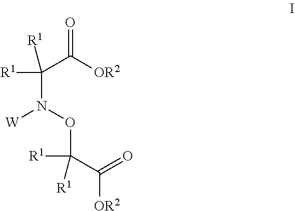
or a salt thereof, wherein:
W is an aryl, wherein the aryl is optionally substituted with one or more groups independently selected from the group consisting of halo, —OH, (C₁-C₄)alkyl, and —C(═O)Ph wherein the —C(═O)Ph is optionally substituted with halo, —OH, or (C₁-C₄)alkyl;
each R¹ is independently (C₁-C₄)alkyl; and
each R² is independently (C₁-C₆)alkyl;
provided the compound is not:

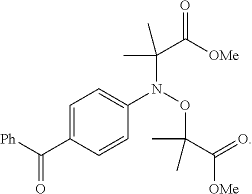 [SMILES: COC(=O)C(C)(C)ON(c1ccc(C(=O)c2ccccc2)cc1)C(C)(C)C(=O)OC]

2. The photopolymerization initiator of claim 1, wherein each R¹ is methyl.

3. The photopolymerization initiator of claim 1, wherein each R² is methyl.

4. The photopolymerization initiator of claim 1, wherein W is naphthalenyl or anthracenyl, wherein the naphthalenyl or anthracenyl is optionally substituted with one or more groups independently selected from the group consisting of halo, —OH, and (C₁-C₄)alkyl.

5. The photopolymerization initiator of claim 1, wherein W is naphthalenyl or anthracenyl, wherein the naphthalenyl or anthracenyl is optionally substituted with one or more —OH.

6. The photopolymerization initiator of claim 1, that is:

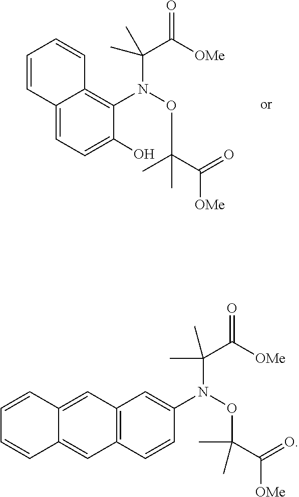 [SMILES: COC(=O)C(C)(C)ON(c1c(O)ccc2ccccc12)C(C)(C)C(=O)OC ; COC(=O)C(C)(C)ON(c1ccc2cc3ccccc3cc2c1)C(C)(C)C(=O)OC]

7. A photopolymerizable composition comprising the photopolymerization initiator of claim 1.

8. The photocurable composition of claim 7 comprising at least one polymerizable compound.

9. A method to polymerize a polymerizable compound or mixture of two or more different polymerizable compounds to provide a polymer, comprising contacting the polymerizable compound or the mixture of two or more different polymerizable compounds with a photopolymerization initiator as described in claim 1 or a photopolymerization initiator of formula I: 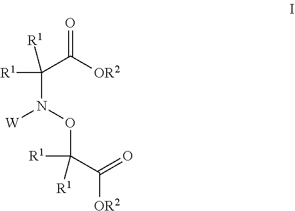
wherein:
W is an aryl, wherein the aryl is optionally substituted with one or more groups independently selected from the group consisting of halo, —OH, (C₁-C₄)alkyl, and —C(═O)Ph wherein the —C(═O)Ph is optionally substituted with halo, —OH, or (C₁-C₄)alkyl;
each R¹ is independently (C₁-C₄)alkyl; and
each R² is independently (C₁-C₆)alkyl
to provide the polymer.

10. The method of claim 9, wherein the polymerizable compound(s) are capable of being polymerized by a radical.

11. The method of claim 9, wherein the polymerizable compound(s) comprise a site of unsaturation.

12. The method of claim 9, wherein the polymerizable compound(s) comprise an unsaturated carbon-carbon double bond.

13. The method of claim 9, wherein the polymerizable compound(s) is an acrylate, diacrylate, methacrylate, dimethacrylate, acrylamide, or bisacrylamide monomer or mixture thereof.

14. The method of claim 13, wherein the acrylate monomer is an alkyl acrylate (e.g., hexyl, pentyl, butyl, propyl ethyl, and methyl acrylate), alkyl methacrylate, bisphenol A epoxy diacrylate, or tri(propylene glycol) diacrylate.

15. The method of claim 9, wherein the photopolymerization initiator and the polymerizable compound(s) are exposed to UV light.

16. A polymer obtained by the method of claim 9.
